(19) United States
(12) Patent Application Publication
(10) Pub. No.: US 2024/0139351 A1
(43) Pub. Date: May 2, 2024

(54) TARGETING SYSTEM WITH IMPROVED UPTAKE

(71) Applicant: CORETAG IP B.V., Rotterdam (NL)

(72) Inventors: Carel Jan VOGELS, Rotterdam (NL); Markwin Hendrik MARING, Rotterdam (NL)

(73) Assignee: CORETAG IP B.V., Rotterdam (NL)

(21) Appl. No.: 18/547,986

(22) PCT Filed: Feb. 23, 2022
(86) PCT No.: PCT/EP2022/054575

§ 371 Date: Aug. 25, 2023

(30) Foreign Application Priority Data
Feb. 25, 2021 (NL) N2027653

(51) Int. Cl. A61K 51/04; A61K 41/00; A61K 49/00; A61P 35/00

(52) U.S. Cl. CPC A61K 51/0497; A61K 41/0052; A61K 49/0002; A61K 49/0032; A61K 49/0052; A61P 35/00

(57) ABSTRACT

The invention relates to a targeting system with improved uptake, the use thereof as a medicament, such as for cancers, to a dosage comprising the targeting system, the use thereof in therapy, the use thereof in treatment, and the use thereof in diagnosis or imaging. In particular the invention relates to systems targeting necrotic cells.

### FIELD OF THE INVENTION

The invention relates to a targeting system with improved uptake, the use thereof as a medicament, such as for cancers, to a dosage comprising the targeting system, the use thereof in therapy, the use thereof in treatment, and the use thereof in diagnosis or imaging. In particular the invention relates to systems targeting necrotic cells.

### BACKGROUND OF THE INVENTION

The present invention relates to targeting of necrotic cells with canines. Targeting of necrosis is specific and unique because necrosis only appears in pathological conditions of cell death due to an insufficient blood supply and thus a lack of oxygen, trauma or due to direct cytotoxic agents or other cancer treatments like radiotherapy or photo dynamic therapy. This is in contrast to apoptotic cell death, which occurs continuously during tissue turnover. Therefore apoptotic cell death, in contrast to necrotic cell death, is not useable for targeting diseases characterized by necrosis as found for instance in case of tumours (rapidly growing tumours spontaneously develop necrotic cores), trauma, infarcts, osteoarthritis, diabetes, arteriosclerotic plaques, burns, certain bacterial infections, etc. The present compounds specifically bind to intracellular proteins.

Further details on background may be obtained from previously filed applications WO 2013/119111 A1, WO 2013/119114 A1, WO 2014/123418 A1, and WO 2020/197397 A1 which documents and their contents are hereby incorporated by reference.

In view of the disclosures of these documents it has found that drug uptake is somewhat hampered, such as by blood constituents interacting with the targeting systems thereof.

Further background art relates to the following documents. US 2016/263249 A1 recites near infrared fluorescent contrast bioimaging agents and methods of use thereof in the medical field, particularly in in vitro diagnostics, in vivo diagnostics, and image-guided surgery, such the use of certain compounds for labelling or likewise contrast agent. Such compounds may be conjugated. Such uses are widely known. Stammes et al., in various documents (Molecular Imaging&Biology, Vol. 18, No. 6, 2016-06-08, p. 905-915, Radiotherapy and Oncology, Vol. 111, May 2015, p. 5124-5125, and Frontiers in Oncology, Vol. 6, 2016-10-21, p. 1-11) recites cyanine-based SPECT-probes for imaging, the use of a specific cyanine for detecting effectiveness of radiotherapy, and the use of a specific cyanine-conjugate for monitoring induced tumor cell death by radiation therapy. Fernandes et al. in Biomedicine&Pharmacotherapy, Vol. 95, Nov. 2017, p. 469-476) recites the role of radionuclide probes for monitoring anti-tumor drug efficacy in a review paper. Zhang et al. in Photochemistry and Photobiology, Vol. 81, Nov. 2005, p. 1499-1504, recites spectral properties of imaging agents with a chelator metal complex, such as a chelator coupled to a radionuclide for PET or SPECT imaging, such as for diagnostic information. These articles are more concerned with imaging/monitoring than targeting or treating. Also, the radionuclide probes are typically used as separate entities.

WO 2016/106324 A1 recites a dye-drug conjugate for preventing, treating, or imaging cancer which may comprise a hydrogen, a therapeutic agent, or an imaging moiety, wherein the therapeutic agent is selected from the group consisting of a platinum-based therapeutic agent, a small molecule therapeutic agent, a peptide, a protein, a polymer, an siRNA, a microRNA, and a nanoparticle, wherein the imaging is a radio-isotope selected from the group consisting of F¹⁸, I¹²⁵, I124 I¹²³, I¹³¹, and small molecule labeled with any of these isotopes, or wherein the imaging moiety is a chelator-complexed radioactive isotope, wherein the radioactive isotope is selected from the group consisting of Cu⁶⁴, In¹¹¹, Tc^{99m}, Ga⁶⁸, Lu¹⁷⁷, Zo⁸⁹, Th²²⁷ and Gd¹⁵⁷. WO2021/138390 A1, published after the filing date of the present application, recites compositions and methods for the detection, and treatment of cancer. Specifically, the compositions of the present technology include multimodal fluorine-cyanine-DOTA-hapten compositions that may be complexed with a radioisotope (e.g., 175Lu). Also disclosed herein are methods of using the fluorine-cyanine-DOTA-hapten compositions of the present technology in diagnostic imaging as well as pretargeted radioimmunotherapy.

The present invention does not relate to DNA binding. DNA-binding molecules are generally considered unsuitable for use in humans due the high chance that such molecules are toxic and possibly also mutagenic/carcinogenic.

It is noted that all proteins comprise amines. Amine reactive molecules will bind to any protein without large differences in affinity. In biological samples, such as blood and tissue, there is a vast amount of extracellular protein present. Selectivity for e.g. dead cells in the sample cannot therefore be achieved. In vivo, strong covalent binding to proteins will result in only very slow clearance from the body, which increases the chance of adverse effects.

It is an object of the present invention to overcome one or more disadvantages of the compositions of the prior art and to provide alternatives to current compositions for diagnosis and treatment of cancers and other diseases involving necrotic cell death, with-out jeopardizing functionality and advantages.

### SUMMARY OF THE INVENTION

It has now been found that drug uptake can be improved by providing a drug up-take molecule in the present targeting molecule. Interaction between blood constituents and the present targeting system is limited thereby. In fact, the pK of uptake is typically at least an order of magnitude better. The present invention relates to a system comprising a targeting system for binding to necrotic cells being a cyanine, preferably non-toxic cyanines, wherein the cyanine is attached to a linker,

wherein the linker is attached to a drug uptake improving molecule, and wherein the drug uptake improving molecule is attached to a chelator according to claim 1.

The present system comprises at least four entities, the entities being joined or linked, such as by a chemical or physical bond, each entity serving a distinct function within the system. More than one cyanine, more than one linker, more than one chelator, and more than one drug uptake improving molecule may be present in the present system; typically not more than two of the foregoing entities are present.

The present targeting molecule is very selective and very specific in binding to necrotic cells. It is non-activated, is capable of non-covalently binding to intracellular proteins when the membrane integrity of a cell is lost, and does not significantly interact with DNA. For the present invention three specific cyanines are selected, namely 800CW, 800RS, and ZW-800. These molecules are found to have a high affinity towards the intracellular proteins, including tubulin and actin. In addition, these cyanines are found to be cleared in the human body specifically well. As a result, very low concentrations c.q. amounts of the present system may be used to provide advantageous effects thereof. It is noted that apoptosis specific probes, in contrast to necrosis specific probes will also target healthy tissue as apoptosis is involved in normal tissue turnover. The present cyanine is attached to a second entity, namely a linker, wherein the linker may even be a relatively small molecule, the linker being attached to the present drug uptake improving molecule, wherein the drug uptake improving molecule is selected from aromatic compounds comprising a residue or group selected from amino acid residues, which latter molecule is attached to the chelator, wherein the chelator is typically selected from DOTA and NOTA comprising compounds (DOTA: 1,4,7,10-Tetraazacy-clododecane-1,4,7,10-tetraacetic acid and NOTA: 1,4,7-triazacyclononane-N,N′,N″-tri-acetic acid). Attached to the chelator a further entity may be attached, such as a radionuclide. The present system is found to be effective as a medicament, also referred to as drug, such as for cancers, in diagnosis, etc. The drug typically relates to a substance intended for use in the diagnosis, therapy, such as radiotherapy, hyperthermia therapy, and radio frequency ablation (RFA) therapy, cure, treatment, or prevention of the aforementioned diseases. For diagnosis relatively short-lived radionuclides (half-life between brackets) may be used, such as ⁶⁴Cu (12.7 h), ⁶⁷Cu (61.8 h), ⁶⁶Ga (9.5 h), ⁶⁷Ga (3.3 d), ⁶⁸Ga (1.2 h), ⁷²Ga (14.1 h), ⁷³Ga (4.9 h), ⁸⁹Zr (78.4 h), ⁸⁷Y (3.4 d), ⁹⁰Y (2.7 d), ¹¹¹In (2.8 d), ¹²³I (13 h), ¹²⁴I (4.2 d), ¹³¹I (8.0 d), ¹⁵⁹Gd (18.5 h), ²¹¹Pb (36 min), ²⁰⁹Pb (3.2 h), ²¹⁴Pb (27 min), and ¹⁷⁷Lu (6.6 d), whereas for treatment relatively long-lived radionuclides may be used, such as ⁸⁸Zr (83.4 d), ⁹⁵Zr (64.0 d), ⁸⁸Y (106.6 d), ⁹¹Y (58.5 d), ¹¹⁴In (>>1 y), ¹²⁵I (59.4d), ¹³¹I (8.0 d), ¹⁵²Gd(>>1 y), ¹⁵³Gd (240 d), ²¹²Pb (10.6 h), ²²⁵Ac (10 d), and ¹⁷³Lu (1.37 y).

It has been found in earlier research by the present inventors that the targeting molecule specifically binds non-covalently to intracellular proteins such as actin, which are only available for the targeting molecule when the membrane integrity of a cell is lost, that is in case of a necrotic cell. For optimal targeting, the necrotic cells, are preferably in an early stage of necrosis, such as cells that have been dead for less than a few days, preferably less than half a day, such as a few hours, such as 2 hours, such as just dead cells. Characteristics of targeting molecules of the present invention, responsible for their effective targeting and/or safety, are that they are cell membrane impermeant, i.e. they cannot (significantly) cross the cell membrane of healthy cells; they are non-activated; they are capable of non-covalently binding to intracellular proteins (their target molecules); and, they are not capable/do not significantly bind to DNA (or RNA).

The present system relates to non-toxic small molecules, having an ability to bind to necrotic cells and tissue. These molecules do not interact with DNA, i.e. these are not toxic or mutagenic. Typically, the present system has a wide-spread biodistribution, cross the blood-brain barrier, do not bind to cell-surface proteins and are sufficiently stable.

In an example crucial time is saved during cancer treatment. The present system instantly provides relevant data on the efficacy of the administered chemotherapeutic drug. An advantage of the present system is that it informs within 24-36 hours after a start of chemotherapy whether or not the therapy is effective. The use will prevent patients from enduring heavy treatments without clinical benefits, which is regarded a major improvement in cancer therapy. The use will result in cost savings. It is noted that unfortunately a (positive) prior art response rate to chemotherapy is limited to 20-35%. Further, the number of treatment cycles is typically limited to a maximum of four due to limitations of the human body. In other words, it is crucial to identify a suitable therapy right from the start, before starting a cycle. By identifying at an early stage of treatment if a treatment is effective the treatment per se can be carried out if considered effective and can be skipped if considered not effective. In the case of a not effective treatment a second treatment can be started likewise. Such can be repeated a further number of times. Once a treatment is considered effective a cycle of chemotherapy can be initiated. It is noted that the identification can be repeated for every and any chemotherapy.

The present system also prevents over-treatment, saving on medicines used to prevent side-effects of cytotoxic drugs.

With the present system there is no need to culture harvested tumour cell e.g. in order to perform tests, which can take a relatively long period of time e.g. a week. The necrotic cells are marked in their natural environment, which e.g. reduces a risk of human errors.

Targeting molecules of the present invention have been found to bind to dead (necrotic) cells very selectively.

As mentioned earlier it has been found by the present inventors that necrotic cells and/or necrosis in general are attractive targets. Such relates to the observation that regions of necrotic cells are typically present in cancers (tumours), e.g. due to an insufficient blood supply and thus lack of oxygen, and in (or result from) diseases involving necrotic cell death. It is noted that regions of necrotic cells are not typically found inside of healthy tissue.

With regards to cancer, once a tumour has been identified, further necrotic cell death can be induced intentionally (for instance by local irradiation, photo dynamic or local thermal therapy and/or focused ultrasound) in part of the tumour to provide a larger target for the composition. Furthermore, wherein the present composition is used for therapeutic purposes, the number of necrotic cells will increase as the therapy progresses thus resulting in dose amplification as a function of time.

A useful discussion on the classification of cell death can be found in Kroemer et al, Cell Death and Differentiation, 2005, 12, 1463. In the present application, necrotic cells are taken as cells whose plasma membrane has lost integrity. A person of skill in the art is able to determine whether a plasma membrane is intact i.e. integral, such as through using fluorescent dyes, such as using commercial amine reactive dyes. The selectivity of the composition of the present invention for dead cells, i.e. cells whose plasma membrane has lost integrity, has been demonstrated in in vitro tests.

The term selectively indicates that the targeting compound has a higher affinity for necrotic cells than for healthy cells (thus the targeting molecules may target necrotic cells). Such can be determined in an in vitro assay as per the examples herewith, or e.g. by flow cytometry; in both methods, co-staining may be used e.g. using commercial live-dead cell staining kits. In simple terms, selective binding in the present invention indicates that for a given population of cells comprising necrosis and healthy cells the number of targeting molecules bound to necrotic cells is at least one order of magnitude higher than the number of targeting molecules bound to healthy cells, typically a few orders of magnitude, and preferably 6 or more orders of magnitude, such as 9 orders.

Non-activated cyanines are cyanines that are non-reactive towards e.g. amines and thiols. Non-activated cyanines cannot significantly (reaction is thermodynamically unfavourable) bind to dead cells (functional groups of molecules thereof) through covalently attaching to amines, thiols or other reactive functional groups present on molecules found inside of cells. That is to say that selective binding to necrotic cells in the context of the present invention is not through covalent bonding, but rather through non-covalent binding via the cyanine core structure and not through the side chains to which the active groups are attached. The term activated cyanine is known to a person of skill in the art and includes e.g. carboxylic acids activated as esters, N-hydroxy succinimide esters, maleimides, acyl chlorides, SDS esters, etc. Non-activated cyanines include e.g. cyanines comprising carboxylic acid functions i.e. the carboxylic acid is not activated.

In a second aspect the present invention relates to a use as a medicament, for use in therapy, in particular in chemotherapy, in immune therapy, in radio-therapy, and in proton-therapy, for use in treatment, such as in treatment of an infarct, such as ischemia of heart or brain, such as for use as a drug for pancreas cancer, for use as a drug for lung cancer, for use as a drug for colorectal cancer, for use as a drug for neck, pharynx or larynx cancer, for use as a drug for glioblastoma cancer, for use as a drug for esophagus cancer, for use as a drug for brain cancer, for use as a drug for myometrium cancer, for use as a drug for ovarium cancer, and for use as a drug for Gastrointestinal stromal cancer, or tumours thereof, or metastases thereof, or

for use in diagnosis or for use in imaging, such as in magnetic resonance imaging (MRI), computed tomography (CT), Single-photon emission computed tomography (SPECT), and fluorescence,

or for targeting necrotic cells, such as induced necrotic cells or spontaneous necrotic cells, or

for targeting inflammation, such as inflammatory cells, such in Croons disease, and colitis ulcerous.

In a further aspect the present invention relates to a use of a dosage according to the invention in an in vivo method for thermal-therapy, the treatment comprising an increase of an internal temperature of the living tumor cells, such as to an internal temperature of at least 42° C.

Advantages of the present description are detailed throughout the description.

### DETAILED DESCRIPTION

It is noted that examples given, as well as embodiments are not considered to be limiting. The scope of the invention is defined by the claims.

In a first aspect the present invention relates a targeting system according to claim 1. Upon testing especially the above cyanines have been found to be very suitable, e.g. in terms of selectivity. Advantageously, cyanines of the invention having a negative charge have been found to bind preferentially to intracellular proteins in the presence of other cell components such as e.g. DNA and RNA. That is to say, cyanines of the invention having a negative charge show in general no significant binding to DNA or RNA. In an exemplary embodiment of the present system the targeting molecule is therefore neutral or negatively charged, wherein neutral cyanines are also found to perform well.

The present drug uptake improving molecule is selected from aromatic compounds, preferably phenyl or naphthyl comprising compounds. These aromatic compounds, and in particular the smaller ones, have been found very effective in improving drug uptake and binding to necrotic cells.

The present drug uptake improving molecule is selected from aromatic compounds comprising a residue or group selected from amino acid residues, wherein the amino acid residue is preferably selected from Alanine, Arginine, Asparagine, Aspartic acid, Cysteine, phenylalanine, Glutamine, Glutamine acid, Glycine, Histidine, Isoleucine, Leucine, Lysine, Methionine, Proline, Pyrroline, Selenocysteine, Serine, Threonine, Tryptophan, Tyrosine, and Valine, preferably an amino acid with a hydrophobic side chain, such as alanine, isoleucine, leucine, methionine, phenylalanine, tryptophan, tyrosine, and valine, from halides, such as chloride, fluoride, iodide, and bromide, preferably chloride, such as 2-naphthylalanine, 3-(2-Naphthyl)-D-alanine (CAS 76985-09-6), 3-(2-Naphthyl)-L-alanine, iodophenyl, chlorophenyl, and fluoro-phenyl. It is also found that, when using these amino acid residues, the amino acid residues themselves can act as a linker. Also more than one amino acid residue may be used.

In an exemplary embodiment of the present system the linker is selected from poly(ethylene)glycol (PEG) linkers, preferably PEG-linkers with n in H—[O—CH₂—CH₂]ₙ—OH from 3-30, more preferably with n from 4-6. Especially relatively short linkers are found to perform well.

In an exemplary embodiment of the present system the chelator comprises DOTA, NOTA, DOTAGA, NOTAGA, or a combination thereof.

In an exemplary embodiment of the present system the targeting molecule is neutral or negatively charged.

In an exemplary embodiment of the present system the cyanine (tetramethyl-indo(di)-carbocyanines) is selected from Streptocyanines, hemicyanines, closed cyanines, neutrocyanines, merocyanines, azacyanines, and apocyanines, preferably from CW-800, 800RS and ZW-800, and combinations thereof, A relatively broad range of cyanines can be used in the present system.

In an exemplary embodiment of the present system at least one functional end group of the cyanine is protected, preferably at least two or three functional end groups, wherein the at least one end group is preferably selected from sulfonates, carboxylates, and hydroxyls, wherein protection is preferably by forming an ester with an alcohol, wherein the alcohol is preferably selected from C₁-C₅ alcohols. This is found to improve the targeting and uptake properties of the present system, such as an improved lipophilic behaviour.

In an exemplary embodiment of the present system attached to the chelator a radionuclide selected from Cu, In, Gd, Ga, Lu, Y, I, Pb, Ac, and Zr, and combinations thereof, or

attached to the chelator a compound selected from a ferromagnetic compound, such as Fe, Co, Ni, Gd, and combinations thereof, from an alloy comprising magnetic components (A,B), wherein component A and/or component B comprise(s) at least one magnetic material selected from Group 3-12, Period 4-6 elements, such as Fe, Co, Ni, Gd, and combinations thereof, such as FePd, FeCo and FePt, and/or wherein component A and/or component B comprise(s) a material selected from lanthanoids, scandium, yttrium, and combinations thereof, such as from Sc, Y, Sm, Gd, Dy, Ho, Er, Yb, Tb, such as Tb, and combinations thereof, and from ferrimagnetic compounds, and alloys from ferrimagnetic compounds

In an exemplary embodiment of the present system the targeting molecule-chelator is selected from 800CW-DOTA, ZW800-DOTA, 800RS-DOTA, 800CW-NOTA, ZW800-NOTA, 800RS-NOTA, or comprising said molecule-chelator, and combinations thereof. These combinations of cyanine/chelator are found to be of particular interest in clinical trials.

In an exemplary embodiment of the present system

the radionuclide is selected from a group consisting of ⁶⁴Cu, ⁶⁷Cu, ⁶⁷Ga, ⁶⁸Ga, ⁷⁰Ga, ⁷²Ga, ⁸⁹Zr, ⁹⁰Y, ⁹⁵Zr, ¹¹¹In, ¹¹⁴In, ¹²³I, ¹²⁴I, ¹⁵³Gd, ¹⁵⁹Gd, ¹⁷⁷Lu, ²⁰⁹Pb, ²¹¹Pb, ²¹²Pb, ²¹⁴Pb, and ²²⁵AC and combinations thereof, wherein the radionuclide is optionally present as a cation, such as with a valence of 0, 1, 2, 3, or 4, such as Ac³⁺, Cu⁺, Cu²⁺, Cu³⁺, Cu⁴⁺, Ga⁺, Ga²⁺, Ga³⁺, Gd⁺, Gd²⁺, Gd³⁺, I⁺, I³⁺, In⁺, In²⁺, In³⁺, Lu³⁺, Zr⁺, Zr²⁺, Zr³⁺, Zr⁴⁺, Pb²⁺, Pb⁴⁺, Y²⁺, and Y³⁺.

Especially these radionuclides are found to provide sufficient action within relative short time frames, with limited side-effects.

In an exemplary embodiment of the present system

the chelator-radionuclide is selected from ⁶⁴Cu_{y}-DOTA, ⁶⁷Cu_{y}-DOTA, ⁶⁷Ga_{y}-DOTA, ⁶⁸Ga_{y}-DOTA, ⁷⁰Ga_{y}-DOTA, ⁷²Ga_{y}-DOTA, ⁸⁹Zr_{y}-DOTA, ⁹⁰Y_{y}-DOTA, ⁹⁵Zr_{y}-DOTA, ¹¹¹In_{y}-DOTA, ¹¹⁴In_{y}-DOTA, ¹²³I_{y}-DOTA, ¹²⁴I_{y}-DOTA, ¹⁵³Gd_{y}-DOTA, ¹⁵⁹Gd_{y}-DOTA, ¹⁷⁷Lu_{y}-DOTA, ²¹²Pb-DOTA, ²²⁵Ac-DOTA ⁶⁴Cu_{y}-NOTA, ⁶⁷Cu_{y}-NOTA, ⁶⁷Ga_{y}-NOTA, ⁶⁸Ga_{y}-NOTA, ⁷⁰Ga_{y}-NOTA, ⁷²Ga_{y}-NOTA, ⁸⁹Zr_{y}-NOTA, ⁹⁰Y_{y}-NOTA, ⁹⁵Zr_{y}-NOTA, ¹¹¹In_{y}-NOTA, ¹¹⁴In_{y}-NOTA, ¹²³I_{y}-NOTA, ¹²⁴I_{y}-NOTA, ¹⁵³Gd_{y}-NOTA, ¹⁵⁹Gd_{y}-NOTA, ¹⁷⁷Lu_{y}-NOTA, ²¹²Pb-NOTA, ²²⁵Ac-NOTA, and combinations thereof, wherein y∈[1,2,3,4].

These combinations of radionuclide/chelator are found to be of particular interest in clinical trials.

In an exemplary embodiment of the present system

the combination of cyanine and radionuclide is selected from ⁶⁴Cu/800RS, ⁶⁷Cu/800RS, ⁶⁷Ga/800RS, ⁶⁸Ga/800RS, ⁷⁰Ga/800RS, ⁷²Ga/800RS, ⁸⁹Zr/800RS, ⁹⁰Y/800RS, ⁹⁵Zr/800RS, ¹¹¹In/800RS, ¹¹⁴In/800RS, ¹²³I/800RS, ¹²⁴I/800RS, ¹⁵³Gd/800RS, ¹⁵⁹Gd/800RS, ¹⁷⁷Lu/800RS, ²¹²Pb/800RS, ²²⁵Ac/800RS, ⁶⁴Cu/800CW, ⁶⁷Cu/800CW, ⁶⁷Ga/800CW, ⁶⁸Ga/800CW, ⁷⁰Ga/800CW, ⁷²Ga/800CW, ⁸⁹Zr/800CW, ⁹⁰Y/800CW, ⁹⁵Zr/800CW, ¹¹¹In/800CW, ¹¹⁴In/800CW, ¹²³I/800CW, ¹²⁴I/800CW, ¹⁵³Gd/800CW, ¹⁵⁹Gd/800CW, ¹⁷⁷Lu/800CW, ²¹²Pb/800CW, ²²⁵Ac/800CW, ⁶⁴Cu/ZW800, ⁶⁷Cu/ZW800, ⁶⁷Ga/ZW800, ⁶⁸Ga/ZW800, ⁷⁰Ga/ZW800, ⁷²Ga/ZW800, ⁸⁹Zr/ZW800, ⁹⁰Y/ZW800, ⁹⁵Zr/ZW800, ¹¹¹In/ZW800, ¹¹⁴In/ZW800, ¹²³I/ZW800, ¹²⁴I/ZW800, ¹⁵³Gd/ZW800, ¹⁵⁹Gd/ZW800, ¹⁷⁷Lu/ZW800, ²¹²Pb/ZW800, ²²⁵Ac/ZW800, and combinations thereof.

In an exemplary embodiment the present system is selected from ⁶⁴Cu_{y}-DOTA-800RS, ⁶⁷Cu_{y}-DOTA-800RS, ⁶⁷Ga_{y}-DOTA-800RS, ⁶⁸Ga_{y}-DOTA-800RS, ⁷⁰Ga_{y}-DOTA-800RS, ⁷²Ga_{y}-DOTA-800RS, ⁸⁹Zr_{y}-DOTA-800RS, ⁹⁰Y_{y}-DOTA-800RS, ⁹⁵Zr_{y}-DOTA-800RS, ¹¹¹In_{y}-DOTA-800RS, ¹¹⁴In_{y}-DOTA-800RS, ¹²³I_{y}-DOTA-800RS, ¹²⁴I_{y}-DOTA-800RS, ¹⁵³Gd_{y}-DOTA-800RS, ¹⁵⁹Gd_{y}-DOTA-800RS, ¹⁷⁷Lu_{y}-DOTA-800RS, ²¹²Pb_{y}-DOTA-800RS, ²²⁵Ac_{y}-DOTA-800RS, ⁶⁴Cu_{y}-DOTA-800CW, ⁶⁷Cu_{y}-DOTA-800CW, ⁶⁷Ga_{y}-DOTA-800CW, ⁶⁸Ga_{y}-DOTA-800CW, ⁷⁰Ga_{y}-DOTA-800CW, ⁷²Ga_{y}-DOTA-800CW, ⁸⁹Zr_{y}-DOTA-800CW, ⁹⁰Y_{y}-DOTA-800CW, ⁹⁵Zr_{y}-DOTA-800CW, ¹¹¹In_{y}-DOTA-800CW, ¹¹⁴In_{y}-DOTA-800CW, ¹²³I_{y}-DOTA-800CW, ¹²⁴I_{y}-DOTA-800CW, ¹⁵³Gd_{y}-DOTA-800CW, ¹⁵⁹Gd_{y}-DOTA-800CW, ¹⁷⁷Lu_{y}-DOTA-800CW, ²¹²Pb_{y}-DOTA-800CW, ²²⁵Ac_{y}-DOTA-800CW, ⁶⁴Cu_{y}-DOTA-ZW800, ⁶⁷Cu_{y}-DOTA-ZW800, ⁶⁷Ga_{y}-DOTA-ZW800, ⁶⁸Ga_{y}-DOTA-ZW800, ⁷⁰Ga_{y}-DOTA-ZW800, ⁷²Ga_{y}-DOTA-ZW800, ⁸⁹Zr_{y}-DOTA-ZW800, ⁹⁰Y_{y}-DOTA-ZW800, ⁹⁵Zr_{y}-DOTA-ZW800, ¹¹¹In_{y}-DOTA-ZW800, ¹¹⁴In_{y}-DOTA-ZW800, ¹²³I_{y}-DOTA-ZW800, ¹²⁴I_{y}-DOTA-ZW800, ^{153 Gd}_{y}-DOTA-ZW800, ¹⁵⁹Gd_{y}-DOTA-ZW800, ¹⁷⁷Lu_{y}-DOTA-ZW800, ²¹²Pb_{y}-DOTA-ZW800, ²²⁵Ac_{y}-DOTA-ZW800, ⁶⁴Cu_{y}-NOTA-800RS, ⁶⁷Cu_{y}-NOTA-800RS, ⁶⁷Ga_{y}-NOTA-800RS, ⁶⁸Ga_{y}-NOTA-800RS, ⁷⁰Ga_{y}-NOTA-800RS, ⁷²Ga_{y}-NOTA-800RS, ⁸⁹Zr_{y}-NOTA-800RS, ⁹⁰Y_{y}-NOTA-800RS, ⁹⁵Zr_{y}-NOTA-800RS, ¹¹¹In_{y}-NOTA-800RS, ¹¹⁴In_{y}-NOTA-800RS, ¹²³I_{y}-NOTA-800RS, ¹²⁴I_{y}-NOTA-800RS, ¹⁵³Gdy-NOTA-800RS, ¹⁵⁹Gd_{y}-NOTA-800RS, ¹⁷⁷Lu_{y}-NOTA-800RS, ²¹²Pb_{y}-NOTA-800RS, ²²⁵Ac_{y}-NOTA-800RS, ⁶⁴Cu_{y}-NOTA-800CW, ⁶⁷Cu_{y}-NOTA-800CW, ⁶⁷Ga_{y}-NOTA-800CW, ⁶⁸Ga_{y}-NOTA-800CW, ⁷⁰Gay-NOTA-800CW, ⁷²Ga_{y}-NOTA-800CW, ⁸⁹Zry-NOTA-800CW, ⁹⁰Y_{y}-NOTA-800CW, ⁹⁵Zr_{y}-NOTA-800CW, ¹¹¹In_{y}-NOTA-800CW, ¹¹⁴Iny-NOTA-800CW, ¹²³Iy-NOTA-800CW, ¹²⁴Iy-NOTA-800CW, ¹⁵³Gd_{y}-NOTA-800CW, ¹⁵⁹Gd_{y}-NOTA-800CW, ¹⁷⁷Lu_{y}-NOTA-800CW, ²¹²Pb_{y}-NOTA-800CW, ²²⁵Ac_{y}-NOTA-800CW, ⁶⁴Cu_{y}-NOTA-ZW800, ⁶⁷Cu_{y}-NOTA-ZW800, ⁶⁷Ga_{y}-NOTA-ZW800, ⁶⁸Gay-NOTA-ZW800, ⁷⁰Ga_{y}-NOTA-ZW800, ⁷²Ga_{y}-NOTA-ZW800, ⁸⁹ Zr_{y}-NOTA-ZW800, ⁹⁰Y_{y}-NOTA-ZW800, ⁹⁵Zr_{y}-NOTA-ZW800, ¹¹¹In_{y}-NOTA-ZW800, ¹¹⁴In_{y}-NOTA-ZW800, ¹²³I_{y}-NOTA-ZW800, ¹²⁴I_{y}-NOTA-ZW800, ¹⁵³Gd_{y}-NOTA-ZW800, ¹⁵⁹Gd_{y}-NOTA-ZW800, ¹⁷⁷Lu_{y}-NOTA-ZW800, ²¹²Pb_{y}-NOTA-ZW800, ²²⁵Ac_{y}-DOTA-ZW800, and combinations thereof, wherein y∈[1,2,3,4].

In an exemplary embodiment the present targeting system is

for use as a medicament, for use in therapy, in particular in chemotherapy, in immune therapy, in radio-therapy, and in proton-therapy, for use in treatment, such as in treatment of an infarct, such as ischemia of heart or brain, such as for use as a drug for pancreas cancer, for use as a drug for lung cancer, for use as a drug for colorectal cancer, for use as a drug for neck, pharynx or larynx cancer, for use as a drug for glioblastoma cancer, for use as a drug for esophagus cancer, for use as a drug for brain cancer, for use as a drug for myometrium cancer, for use as a drug for ovarium cancer, and for use as a drug for Gastrointestinal stromal cancer, or tumours thereof, or metastases thereof, or

for use in diagnosis or for use in imaging, such as in magnetic resonance imaging (MRI), computed tomography (CT), Single-photon emission computed tomography (SPECT), and fluorescence,

or for targeting necrotic cells, such as induced necrotic cells or spontaneous necrotic cells, or

for targeting inflammation, such as inflammatory cells, such in Crohn's disease, and colitis ulcerous.

In a second aspect the present invention relates to a dosage for use as a medicament, such as a drug for treatment of cancers selected from esophagus, pharynx and larynx, lung, brain, Pancreas, colorectal, head neck, glioblastoma, myometrium, ovarium, Gastrointestinal stromal cancer, tumours thereof, or metastases thereof, comprising an effective amount of the system of the invention.

The invention also relates to a method of establishing a dosage, comprising determining a body weight in kg, and multiplying the body weight with 0.1-1000 nMole system according to the invention.

In an example the dosage comprises an amount of 0.1-1000 nMole system/kg body weight, preferably 0.5-500 nMole system/kg body weight, more preferably 1-250 nMole system/kg body weight, even more preferably 2-100 nMole system/kg body weight, such as 5-50 nMole system/kg body weight; such may relate to a dosage of e.g. 0.01-200 mgram. The dosage preferably is provided in a physiological acceptable solution of 1-50 ml. Preferably a kit comprising some (1-50) dosages is provided.

In a further aspect the present invention relates to multiple dosages according to the invention, such as 2-4 dosages, for intermittent application, such as with intervals of 0.2-4 hours, preferably 1-2 hours.

The invention is further detailed by the Examples and accompanying figures, which are exemplary and explanatory of nature and are not limiting the scope of the invention. To the person skilled in the art it may be clear that many variants, being obvious or not, may be conceivable falling within the scope of protection, defined by the present claims.

### SUMMARY OF FIGURES

FIG. 1a-c shows cyanines, FIG. 2a-b chelators, and FIG. 3 complexes, FIGS. 4a-b-5 experimental results, and FIGS. 6-7 targeting systems.

### DETAILS OF THE FIGURES

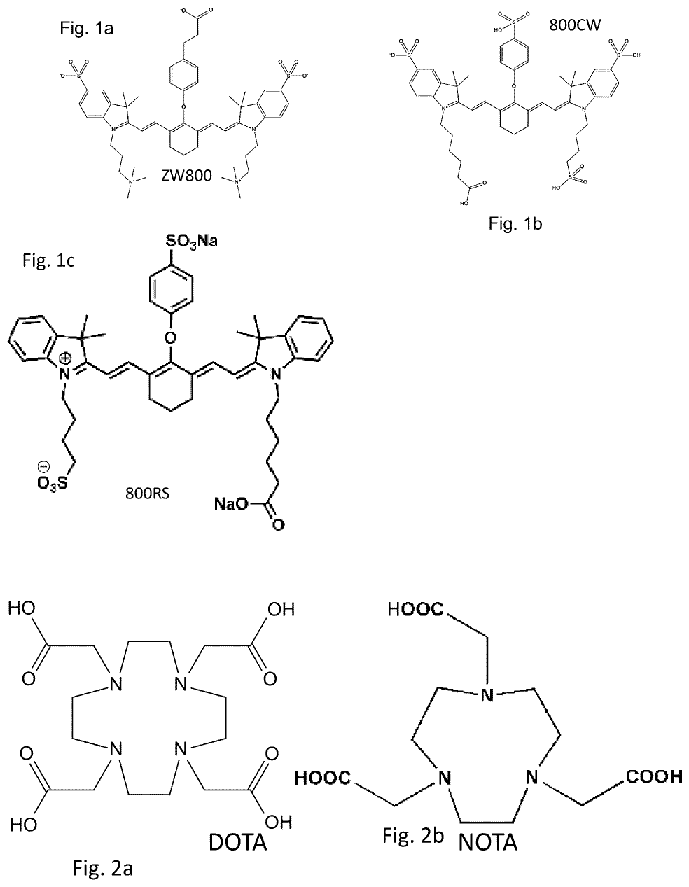

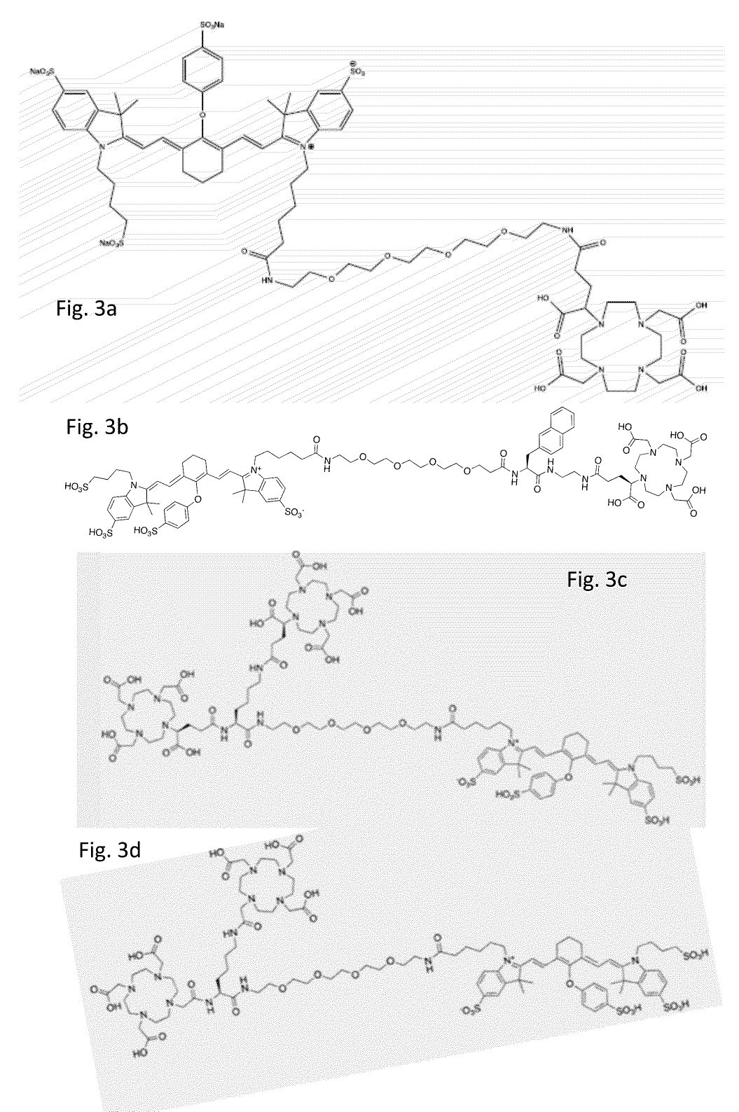

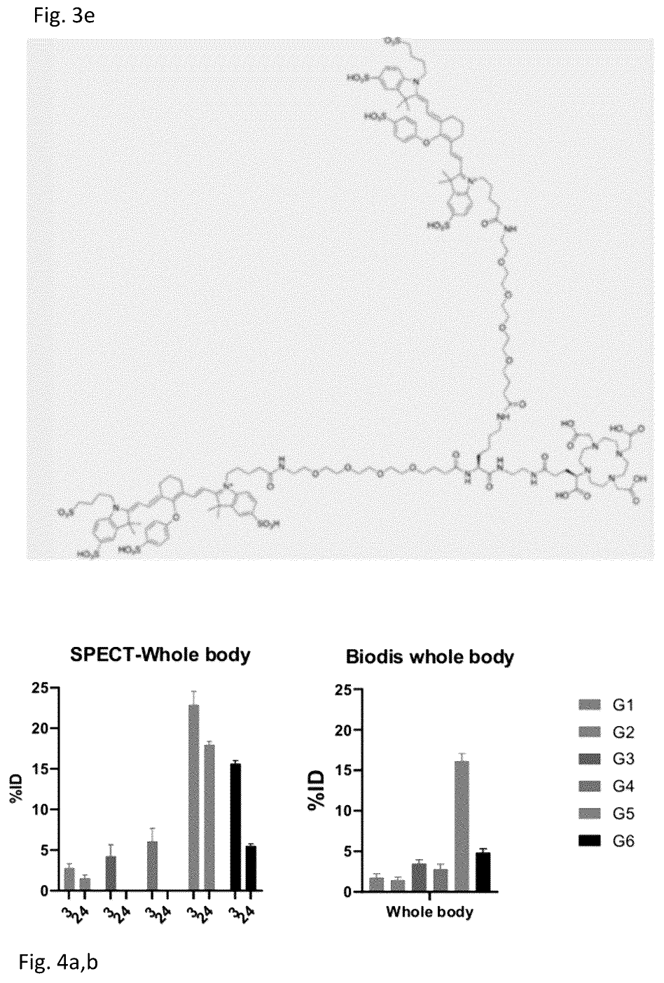

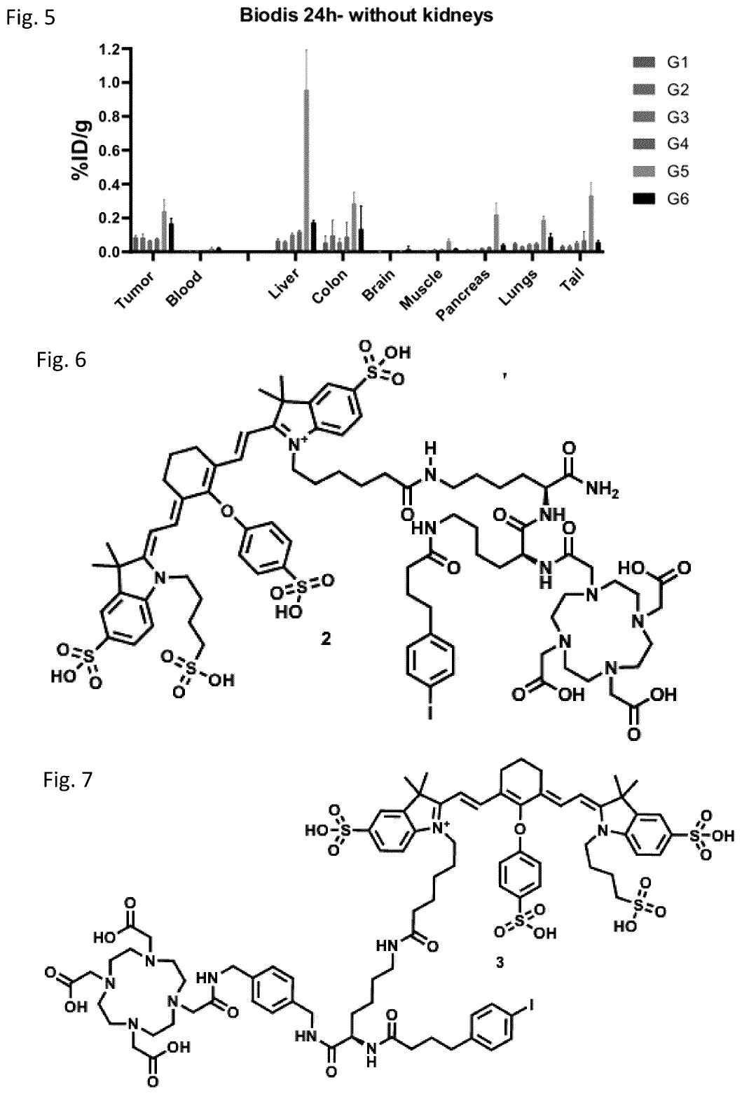

FIG. 1a-c shows chemical structures of various cyanines. FIGS. 1a-c are cyanines according to the invention.

FIG. 2a-b shows chemical structures of DOTA and NOTA.

FIGS. 3a-e show a cyanine-DOTA complexes. In particular, FIG. 3b shows IR800CW-PEG4-NaI-DOTA-GA with chemical formula C₉₁H₁₂₂N₁₀O₂₉S₄, and a total molecular mass of 1948.26, also referred to as component 6. It is obtained in a purity of >95%, it is dark green, and forms an aqueous solution of >1 mM. FIG. 3c shows 2DOT-AGA-LYS-PEG4-CW800 (DOTAGA-K-DOTAGA-PEG4-IR800CW), also referred to as component 4. FIG. 3d shows 2DOTA-LYS-PEG4-CW800 (DOTA-K-DOTA-PEG4-IR800CW), also referred to as component 3. FIG. 3e shows DOTAGA-LYS-(PEG4-CW800)2 (IR800CW-PEG4-K-(IR800CW-PEG4)-DOTAGA), also referred to as component 5. A similar component with only one PEG-CW800 is referred to as component 1 and 2. FIG. 3f shows DOTAGA-LYS-(PEG4-CW800)2 (IRCW800-PEG4-K-(IR800CW-PEG4)-DOTAGA).

FIGS. 4a-b and 5 show experimental results.

FIGS. 6-7 show exemplary targeting systems.

### EXAMPLES

### Clinical Trials

Various clinical trials are performed. For some cancers, namely lung cancer, esophagus cancer, brain cancer, larynx/pharynx cancer, and cancer of the intestines populations of patients are followed during treatment, therapy and diagnosis.

At the University Hospital Tubingen (UKT) various clinical trials are performed with the present targeting system as both diagnostic and therapeutic medicine for esophagus-, brain-, pharynx- and larynx-, lung-, and intestines-cancer. In this clinical trials cancer patients are followed during protocols of diagnosis, treatment and therapy.

For the diagnostic treatment the chelator is labeled with a radionuclide, like Zirconium, Gallium, Gadolinium, or Copper, and intravenously administered to the patient followed by medical imaging with a PET-, SPECT- or MRI-imaging device. Diagnostic treatment of necro-sis results in the detection, localization and quantify of necrotic tissue in the human body.

For the radio-therapeutic treatment the chelator is labeled with a radionuclide like Copper or Lutetium and administered to the patient. The radionuclide radiates the living tumor-cells from within the tumor, as it is bound to the necrotic tumor cores.

For the Hyperthermia-treatment the chelator is labeled with a magnetic molecule like gadolinium and administered to the patient intravenously. As the gadolinium is bound to the necrotic cores in the tumor, the surrounding living tumor is selectively heated with an MRI-imaging device for increasing the tumor cells to 42° C. or more, which leads to increase of living tumor cells.

### Results

For all trials initial results indicate that the present targeting system increases necrosis at a location of the respective cancers.

In addition it has been found that the present system can be used to establish the effect of a further treatment, such as immunotherapy, chemotherapy, proton therapy, and radiotherapy, by using the targeting system to attach to the necrotic cells formed as a result of the further treatment. Such is important, as now in an early stage an effect of the further treatment can be established; if no effect or a small effect is visible, the further treatment can be stopped.

The present targeting system itself is also effective as radiotherapy, as the radionuclide of the system increases local necrosis, as is intended.

As the present system can be used in very low dosage regimes, no side-effects are observed. In addition it has been confirmed that the present system can be cleared form the human body to a high extent.

It is noted that a healthy human body does not contain necrotic tissue, which means that when necrosis is detected by the present targeting system this indicates a pathological situation like stroke, myocardial infarction, osteo-arthritis and/or a aggressively growing tumor. Aggressively growing (malign) tumors and/or metastases develop a necrotic core starting at a diameter of 3mm or more. Detecting necrosis in a human body is relevant because based on this outcome medical professionals can diagnose medical issues, determine treatment options and make medical relevant decisions of which the patient can benefit.

In addition it has been found that the present system can be used to establish the effect of a further treatment, such as immunotherapy, chemotherapy, by using the targeting system to attach to the necrotic cells formed as a result of the further treatment. Such is important, as now in an early stage an effect of the further treatment can be established; if no effect or a small effect is visible, the further treatment can be stopped.

The present targeting system provides a new early stage method to determine the efficacy of the treatment of aggressively growing (malign) tumors. The outcome and results of conventional therapies like chemotherapy and radio-therapy, but also new therapies like immune-, brachy- or proton-therapy is only available after multiple treatments. Moreover, the outcome of chemo- and immunotherapy is only known after multiple treatments over a longer period of time.

Today shrinkage and decrease of volume of the tumor are indications that a treatment is effective. Fact is that in average chemotherapies are costly but only effective in a minority of the cases. With the present targeting system necrosis in the tumor is quantified before (T=0) and shortly after (T=1) the administration of the chemo- or immuno-therapy. The delta of the radioactive signal between T=0 and T=1 reflects the efficacy of the administered therapy. Implementing this application with the present targeting system provides a reduction of the ineffective treatments with 35% or more. Besides improving quality of life by avoiding severe treatments of patients which do not have to suffer from a treatment which does not provide the desired effect, the cost reduction of cancer treatment is tremendous. Cancer treatment (direct and side-effect treatment) represent 75% or more of the total budget for drugs and treatments in an average hospital. Possible reductions in costs of care and medicines in the Western world alone represent at least € 2 Billions.

The present targeting system itself is also effective as radiotherapy, as the radionuclide of the system increases local necrosis by killing living tumor-cells with nuclear radiation, as is intended. It is found that typically a volume of tumor-cells is reduced by 50% or more, and often these are not visible anymore. In addition the present treatment provides sufficient control of the tumor, and limits further development thereof, such that patients require on regular treatment at the most; hence patients can now be treated comparable to other long-term diseases and continue mostly or fully with their lives.

As the present system can be used in very low dosage regimes, the treatment is targeted and limited to the tumor and no side-effects are observed. In addition it has been confirmed that the present system binds to the necrotic core of tumors and the unbound compounds can be cleared from the human body to a high extent within a maximum of 24-hours after administration. This new hyperthermia treatment options provides important new treatment options for non-resectable or non-treatable aggressively growing (malign) tumors. This new targeted in-tumor radiotherapy treatment provides important new options for non-resectable or non-treatable aggressively growing (malign) tumors, such as the ones claimed.

The present targeting system itself is also effective as thermal-therapy, the treatment of the iron-labelled chelator with MRI results in the increase of the internal temperature of the living tumor cells. After the internal temperature of these cells is 42° C., or higher, this treatment results in the death of this tumor cells. This new form of hyperthermia treatment provides important new treatment options for non-receptable or non-treatable aggressively growing (malign) tumors.

Study results of the present targeting system show an increase of dead (effectively treated) tumor in 40-60% of tumors (as is 35% on average for today's chemotherapy). Repetitive treatments with present targeting system for the same tumor show increase of tumor death by 40-60% for each treatment-cycle that can add up to eradicating all of living cells in a tumor.

CORETAG has evaluated the radiolabeling, in vitro binding, pharmacology in tumor animal model, and PET or SPECT imaging with an overall expertise in targeted radiotherapy and companion imaging diagnostic. The aim of this part is to evaluate the biodistribution of the 4 compounds below and compare it to the biodistribution of DOT-AGA-PEG4-CW800 (prior art).

### Radio Labeling

DOTAGA-PEG4-800CW, 2DOTAGA-Lys-PEG4-800CW, 2DOTA-Lys-PEG4-800CW, DOTAGA-Lys-(PEG4-800CW)2 and DOTAGA-(2-Nal)-PEG4-800CW were radiolabeled with ¹¹¹In. The injected activity was about 9.0 MBq.

### Cancer Line

The NCI-H69 cell line used is a human small cell lung cancer carcinoma that was

established from a 55 year-old Caucasian patient3]. The cancer cell line(s) and culture media are provided by Oncodesign.

### Cell Culture Method

Tumor cells were grown at 37° C. in a humidified atmosphere (5% CO2, 95% air). The culture medium will be RPMI1640 medium containing 10% fetal calf serum. NCI-H69 tumor cells are growing as a cell suspension. For experimental use, tumor cells were harvested by centrifugation. Cells were counted and viability assessed using a 0.25% try-pan blue exclusion assay.

### Animals

Thirty-one (31) healthy female SWISS Nude (Crl:NU(Ico)-Foxn1nu) mice, 6-8 weeks old at reception, were obtained from Charles River. The injection volume was 100 μl.

### Tumor Induction

Tumors were induced by subcutaneous injection of 2×107 NCI-H69 cells in 200 μL of RPMI 1640 into the right flank of 31 female animals in the axis of the heart. NCI-H69 tumor cell implantation was performed 24 to 72 hours after a whole-body irradiation with a gamma-source (2 Gy, 60 Co, BioMep, France). The day of tumor induction is considered as D0.

### MRI Material

All imaging experiments were performed on a 4.7T horizontal magnet (PharmaS-can, Bruker Biospin GmbH, Germany) equipped with an actively shielded gradient system. All the MR images were acquired under ParaVision (PV5.1, Bruker Biospin).

### Results

Radioactivity is rapidly cleared from blood (<0.5% ID/g after 2.7 h)

Radioactivity in blood is higher in components 5 and 6.

Remaining radioactivity in whole body after 3 h is higher in components 5 and 6

Remaining radioactivity in whole body after 24 h is higher in component 5 [see FIG. 4a,b). What is found therefore is a longer concentration and a higher concentration in the blood. As a consequence the dosage to the tumor is typically about twice as high for component 6, according to the invention, compared to components 1-4 (prior art). Such is the case for all organs tested, in particular the liver, the colon, the brain, the muscle, the pancreas, the lungs, and in addition the tail (see FIG. 5).

Component 5 is considered to be present in a too high concentration.

To put different, for the biodistribution in organs, the uptake is mainly observed in

kidneys, followed by the uptake in tumors, liver and colon. Uptake increased for component 5 in all organs. An a higher uptake in tumor for components 5 and 6 was observed.

1. A targeting system comprising a targeting molecule for binding to necrotic cells, the targeting molecule being selected from cyanines,
wherein the cyanine is attached to a linker,
wherein the linker is attached to a drug uptake improving molecule,
and wherein the drug uptake improving molecule is attached to a chelator,
wherein the drug uptake improving molecule is selected from aromatic compounds comprising at least one of a residue and group selected from amino acid residues.

2. The targeting system according to claim 1, wherein the drug uptake improving molecule is selected from at least one of phenyl and naphthyl comprising compounds.

3. The targeting system according to claim 1, wherein the amino acid residue of the drug uptake improving molecule is selected from Alanine, Arginine, Asparagine, Aspartic acid, Cysteine, phenylalanine, Glutamine, Glutamine acid, Glycine, Histidine, Isoleucine, Leucine, Lysine, Methionine, Proline, Pyrroline, Selenocysteine, Serine, Threonine, Tryptophan, Tyrosine, and Valine, and from halides.

4. The targeting system according to claim 1, wherein the linker is selected from poly(ethylene)glycol (PEG) linkers.

5. The targeting system according to claim 1, wherein the chelator comprises at least one of DOTA, NOTA, DOTAGA, NOTAGA, and a combination thereof.

6. The targeting system according to claim 1, wherein the targeting molecule is selected from neutral and negatively charged targetin molecules.

7. The targeting system according to claim 1, wherein the cyanine (tetramethylindo(di)-carbocyanines) is selected from Streptocyanines, hemicyanines, closed cyanines, neutrocyanines, merocyanines, azacyanines, and apocyanines, and combinations thereof, and
wherein at least one functional end group of the cyanine is protected.

8. The targeting system according to claims 1, and attached to the chelator a compound selected from at least one of a radionuclide selected from at least one of Cu, In, Gd, Ga, Lu, Y, I, Pb, Ac, and Zr, and combinations thereof, from a ferromagnetic compound, and from an alloy comprising magnetic components (A,B), wherein at least one of component A and component B comprise(s) at least one magnetic material selected from Group 3-12, Period 4-6 elements, and combinations thereof, and wherein component A and component B comprise(s) a material selected from lanthanoids, scandium, yttrium, and combinations thereof, and from ferrimagnetic compounds, and alloys from ferrimagnetic compounds.

9. The targeting system according to of claim 1, wherein the targeting molecule-chelator is selected from 800CW-DOTA, ZW800-DOTA, 800RS-DOTA, 800CW-NOTA, ZW800-NOTA, 800RS-NOTA, and comprising said molecule-chelator, and combinations thereof.

10. The targeting system according to claim 8, wherein the radionuclide is selected from a group consisting of ⁶⁴Cu, ⁶⁷Cu, ⁶⁷Ga, ⁶⁸Ga, ⁷⁰Ga, ⁷²Ga, ⁸⁹Zr, ⁹⁰Y, ⁹⁵Zr, ¹¹¹In, ¹¹⁴In, ¹²³I, ¹²⁴I, ¹⁵³Gd, ¹⁵⁹Gd, ¹⁷⁷Lu, ²⁰⁹Pb, ²¹¹Pb, ²¹²Pb, ²¹⁴Pb, ²²⁵Ac, and combinations thereof, wherein the radionuclide is optionally present as a cation, such as with a valence of 0, 1, 2, 3, or 4.

11. The targeting system according to claim 8, wherein the chelator-radionuclide is selected from ⁶⁴Cu_{y}-DOTA, ⁶⁷Cu_{y}-DOTA, ⁶⁷Ga_{y}-DOTA, ⁶⁸Ga^{y}-DOTA, ⁷⁰Ga_{y}-DOTA, ⁷²Ga_{y}-DOTA, ⁸⁹Zr_{y}-DOTA, ⁹⁰Y_{y}-DOTA, ⁹⁵Zr_{y}-DOTA, ¹¹¹In_{y}-DOTA, ¹¹⁴In_{y}-DOTA, ¹²³I_{y}-DOTA, ¹²⁴I_{y}-DOTA, ¹⁵³Gd_{y}-DOTA, ¹⁵⁹Gd_{y}-DOTA, ¹⁷⁷Lu_{y}-DOTA, ²¹²Pb_{y}-DOTA, ²²⁵Ac_{y}-DOTA, ⁶⁴Cu_{y}-NOTA, ⁶⁷Cu_{y}-NOTA, ⁶⁷Ga_{y}-NOTA, ⁶⁸Ga_{y}-NOTA, ⁷⁰Ga_{y}-NOTA, ⁷²Ga_{y}-NOTA, ⁸⁹Zr_{y}-NOTA, ⁹⁰Y_{y}-NOTA, ⁹⁵Zr_{y}-NOTA, ¹¹¹In_{y}-NOTA, ¹¹⁴In_{y}-NOTA, ¹²³I_{y}-NOTA, ¹²⁴I_{y}-NOTA, ¹⁵³Gd_{y}-NOTA, ¹⁵⁹Gd_{y}-NOTA, ¹⁷⁷Lu_{y}-NOTA, ²¹²Pb-NOTA, ²²⁵Ac_{y}-NOTA, and combinations thereof, wherein y∈[1,2,3,4].

12. The targeting system according to claim 8, wherein the combination of cyanine and radionuclide is selected from ⁶⁴Cu/800RS, ⁶⁷Cu/800RS, ⁶⁷Ga/800RS, ⁶⁸Ga/800RS, ⁷⁰Ga/800RS, ⁷²Ga/800RS, ⁸⁹Zr/800RS, ⁹⁰Y/800RS, ⁹⁵Zr/800RS, ¹¹¹In/800RS, ¹¹⁴In/800RS, ¹²³I/800RS, ¹²⁴I/800RS, ¹⁵³Gd/800RS, ¹⁵⁹Gd/800RS, ¹⁷⁷Lu/800RS, ²¹²Pb/800RS, ²²⁵Ac/800RS, ⁶⁴Cu/800CW, ⁶⁷Cu/800CW, ⁶⁷Ga/800CW, ⁶⁸Ga/800CW, ⁷⁰Ga/800CW, ⁷²Ga/800CW, ⁸⁹Zr/800CW, ⁹⁰Y/800CW, ⁹⁵Zr/800CW, ¹¹¹In/800CW, ¹¹⁴In/800CW, ¹²³I/800CW, ¹²⁴I/800CW, ¹⁵³Gd/800CW, ¹⁵⁹Gd/800CW, ¹⁷⁷Lu/800CW, ²¹²Pb/800CW, ²²⁵Ac/800CW, ⁶⁴Cu/ZW800, ⁶⁷Cu/ZW800, ⁶⁷Ga/ZW800, ⁶⁸Ga/ZW800, ⁷⁰Ga/ZW800, ⁷²Ga/ZW800, ⁸⁹Zr/ZW800, ⁹⁰Y/ZW800, ⁹⁵Zr/ZW800, ¹¹¹In/ZW800, ¹¹⁴In/ZW800, ¹²³I/ZW800, ¹²⁴I/ZW800, ¹⁵³Gd/ZW800, ¹⁵⁹Gd/ZW800, ¹⁷⁷Lu/ZW800, ²¹²Pb/ZW800, ²²⁵Ac/ZW800, and combinations thereof.

13. The targeting system according to claim 8, wherein the combination of cyanine, linker, and radionuclide is selected from ⁶⁴Cu_{y}-DOTA-800RS, ⁶⁷Cu_{y}-DOTA-800RS, ⁶⁷Ga_{y}-DOTA-800RS, ⁶⁸Ga_{y}-DOTA-800RS, ⁷⁰Ga_{y}-DOTA-800RS, ⁷²Ga_{y}-DOTA-800RS, ⁸⁹Zr_{y}-DOTA-800RS, ⁹⁰Y_{y}-DOTA-800RS, ⁹⁵Zr_{y}-DOTA-800RS, ¹¹¹In_{y}-DOTA-800RS, ¹¹⁴In_{y}-DOTA-800RS, ¹²³I_{y}-DOTA-800RS, ¹²⁴I_{y}-DOTA-800RS, ¹⁵³Gd_{y}-DOTA-800RS, ¹⁵⁹Gd_{y}-DOTA-800RS, ¹⁷⁷Lu_{y}-DOTA-800RS, ²¹²Pb_{y}-DOTA-800RS, ²²⁵Ac_{y}-DOTA-800RS, ⁶⁴Cu_{y}-DOTA-800CW, ⁶⁷Cu_{y}-DOTA-800CW, ⁶⁷Ga_{y}-DOTA-800CW, ⁶⁸Ga_{y}-DOTA-800CW, ⁷⁰Ga_{y}-DOTA-800CW, ⁷²Ga_{y}-DOTA-800CW, ⁸⁹Zr_{y}-DOTA-800CW, ⁹⁰Y_{y}-DOTA-800CW, ⁹⁵Zr_{y}-DOTA-800CW, ¹¹¹In_{y}-DOTA-800CW, ¹¹⁴In_{y}-DOTA-800CW, ¹²³I_{y}-DOTA-800CW, ¹²⁴I_{y}-DOTA-800CW, ¹⁵³Gd_{y}-DOTA-800CW, ¹⁵⁹Gd_{y}-DOTA-800CW, ¹⁷⁷Lu_{y}-DOTA-800CW, ²¹²Pb_{y}-DOTA-800CW, ²²⁵Ac_{y}-DOTA-800CW, ⁶⁴Cu_{y}-DOTA-ZW800, ⁶⁷Cu_{y}-DOTA-ZW800, 67^{Ga}_{y}-DOTA-ZW800, ⁶⁸Ga_{y}-DOTA-ZW800, ⁷⁰Ga_{y}-DOTA-ZW800, ⁷²Ga_{y}-DOTA-ZW800, ⁸⁹Zr_{y}-DOTA-ZW800, ⁹⁰Y_{y}-DOTA-ZW800, ⁹⁵Zr_{y}-DOTA-ZW800, ¹¹¹In_{y}-DOTA-ZW800, ¹¹⁴In_{y}-DOTA-ZW800, ¹²³I_{y}-DOTA-ZW800, ¹²⁴I_{y}-DOTA-ZW800, ¹⁵³Gd_{y}-DOTA-ZW800, ¹⁵⁹Gd_{y}-DOTA-ZW800, ¹⁷⁷Lu_{y}-DOTA-ZW800, ²¹²Pb_{y}-DOTA-ZW800, ²²⁵Ac_{y}-DOTA-ZW800, ⁶⁴Cu_{y}-NOTA-800RS, ⁶⁷Cu_{y}-NOTA-800RS, ⁶⁷Ga_{y}-NOTA-800RS, ⁶⁸Ga_{y}-NOTA-800RS, ⁷⁰Ga_{y}-NOTA-800RS, ⁷²Ga_{y}-NOTA-800RS, ⁸⁹Zr_{y}-NOTA-800RS, ⁹⁰Y_{y}-NOTA-800RS, ⁹⁵Zr_{y}-NOTA-800RS, ¹¹¹In_{y}-NOTA-800RS, ¹¹⁴In_{y}-NOTA-800RS, ¹²³I_{y}-NOTA-800RS, ¹²⁴I_{y}-NOTA-800RS, ¹⁵³Gd_{y}-NOTA-800RS, ¹⁵⁹Gd_{y}-NOTA-800RS, ¹⁷⁷Lu_{y}-NOTA-800RS, ²¹²Pb_{y}-NOTA-800RS, ²²⁵Ac_{y}-NOTA-800RS, ⁶⁴Cu_{y}-NOTA-800CW, ⁶⁷Cu_{y}-NOTA-800CW, ⁶⁷Gay-NOTA-800CW, ⁶⁸Ga_{y}-NOTA-800CW, ⁷⁰Gay-NOTA-800CW, ⁷²Ga_{y}-NOTA-800CW, ⁸⁹Zry-NOTA-800CW, ⁹⁰Y_{y}-NOTA-800CW, ⁹⁵Zr_{y}-NOTA-800CW, ¹¹¹In_{y}-NOTA-800CW, ¹¹⁴Iny-NOTA-800CW, ¹²³Iy NOTA-800CW, ¹²⁴Iy-NOTA-800CW, ¹⁵³Gd_{y}-NOTA-800CW, ¹⁵⁹Gd_{y}-NOTA-800CW, ¹⁷⁷Lu_{y}-NOTA-800CW, ²¹²Pb_{y}-NOTA-800CW, ²²⁵Ac_{y}-NOTA-800CW, ⁶⁴Cu_{y}-NOTA-ZW800, ⁶⁷Cu_{y}-NOTA-ZW800, ⁶⁷Ga_{y}-NOTA-ZW800, ⁶⁸Gay-NOTA-ZW800, ⁷⁰Ga_{y}-NOTA-ZW800, ⁷²Ga_{y}-NOTA-ZW800, ⁸⁹Zr_{y}-NOTA-ZW800, ⁹⁰Y_{y}-NOTA-ZW800, ⁹⁵Zr_{y}-NOTA-ZW800, ¹¹¹In_{y}-NOTA-ZW800, ¹¹⁴In_{y}-NOTA-ZW800, ¹²³I_{y}-NOTA-ZW800, ¹²⁴I_{y}-NOTA-ZW800, ¹⁵³Gd_{y}-NOTA-ZW800, ¹⁵⁹Gd_{y}-NOTA-ZW800, ¹⁷⁷Lu_{y}-NOTA-ZW800, ²¹²Pb_{y}-NOTA-ZW800, ²²⁵Ac_{y}-DOTA-ZW800, and combinations thereof, wherein y∈[1,2,3,4].

14. (canceled)

15. A method of treating cancers selected from esophagus cancer, pharynx and larynx carcer, lung cancer, brain cancer, Pancreas cancer, colorectal cancer, head neck cancer, glioblastoma cancer, myometrium cancer, ovarium cancer, Gastrointestinal stromal cancer, tumours thereof, or metastases thereof, comprising administering a dosage of an effective amount of the system of claim 1.

16. (canceled)

17. The method according to claim 15, comprising administering an amount of 0.1-1000 nMole system/kg body weight and wherein the dosage is provided in a physiological acceptable solution of 1-50 ml.

18. The method according to claim 15, comprising administering multiple dosages for intermittent application.

19. (canceled)

20. A method of imaging, wherein imaging is selected from magnetic resonance imaging (MRI), computed tomography (CT), Single-photon emission computed tomography (SPECT), and fluorescence, the method comprising applying the targeting system of claim 1.

21. A method of treating at least one of
an infarct, ischemia of heart, ischemia of brain, pancreas cancer, lung cancer, colorectal cancer, neck cancer, pharynx cancer, larynx cancer, glioblastoma cancer, esophagus cancer, brain cancer, myometrium cancer, ovarium cancer, gastrointestinal stromal cancer, and tumours thereof, and metastases thereof, comprising
applying the targeting system according to claim 1.
